(19) United States
(12) Patent Application Publication
(10) Pub. No.: US 2017/0307578 A1
(43) Pub. Date: Oct. 26, 2017

(54) Meat Product Fat Testing System

(71) Applicant: Daniel Banayan, Encino, CA (US)

(72) Inventor: Daniel Banayan, Encino, CA (US)

(21) Appl. No.: 15/138,472

(22) Filed: Apr. 26, 2016

(51) Int. Cl. G01N 33/12; G01N 5/04; G01G 19/40; G01N 33/03

(52) U.S. Cl. CPC G01N 33/12; G01N 33/03; G01N 5/04; G01G 19/40; A23V 2002/00

(57) ABSTRACT

A meat product fat testing system (MPFTS) for determining the percent fat content of a meat product. The MPFTS includes two embodiments: a fat testing/analyzing unit and a hand-held calculating unit. The fat testing/analyzing unit integrally houses a heating means, meat product weighing means, a fat/moisture analyzer, a microcontroller, and a display. A sample of a meat product is placed into the unit and the unit is activated. The percent fat content of the sample is automatically analyzed and the result shown on the unit's display. The hand-held calculating unit is used with a conventional scale and heat source, which are used to obtain wet and dry weight readings of a meat product sample. The readings are input into the calculating device, which then displays in a window the percent fat content of the sample.

### TECHNICAL FIELD

The invention generally pertains to food testing devices, and more particularly to a meat product fat testing system that includes a mechanical embodiment and an integral electronic embodiment for analyzing and calculating the percent fat content of a meat product.

### BACKGROUND ART

To assist in maintaining a healthy lifestyle, many new products and concepts have been introduced. These can range from activities such as exercising, working out at a gym, dancing and using home exercise equipment. Special foods and recipes for health-conscious eating are also widely used. One of the primary concerns of the health-food industry is the ability to disclose to the consumer the positive or negative nutrition information for a specific food. Most health-conscious individuals are concerned about in the amount of calories, sodium and, especially fat that is present in the food they eat. Supplier and retailers typically also need and want to know information about the food they sell.

This information is especially required in meat products such as beef and pork, which typically have a high percentage of fat. The process of determining the percent fat in meat products often requires considerable time and effort to produce accurate results due to the equipment and steps needed. Obviously, if a device and method are available that are capable of quickly and accurately measuring the percent fat of a meat product, it would be beneficial to consumers especially health-conscious individuals, meat suppliers and retailers.

A search of the prior art did not disclose any literature or patents that read directly on the claims of the instant invention. However, the following U.S. patents are considered related:

| PAT. NO. | INVENTOR | ISSUED |
|---|---|---|
| 5,858,795 | Banayan | 12 Jan. 1999 |
| 4,753,889 | Collins | 28 Jun. 1988 |
| 3,909,598 | Collins, et al | 30 Sep. 1975 |

The U.S. Pat. No. 5,858,795 discloses a device for determining the percent fat of a meat product such as beef or pork. The device is presented in two designs, the first design uses a mechanical device in the form of a slide chart and the second design employs an electronic device which is disclosed in three design configurations. The slide chart includes a stationary member and movable member. When a selected percent moisture figure is located on a percent moisture window the equivalent percent fat figure is displayed on percent fat window. The electronic device consists of either a hand-held electronic calculator, a percent moisture analyzer that is connected to an electronic calculator or an integrated percent moisture and fat analyzer.

The U.S. Pat. No. 4,753,889 discloses a method and an apparatus for quantitatively measuring solids. The apparatus provides rapid and accurate determinations using microwave heating, electronic balance weighing and solvent extracting of solubles. The method involves subjecting a weighed sample to microwave drying for a preselected time, reweighing to obtain the dry weight and determine volatible loss, solvent extracting the dried sample to determine fats, oils and other extractables followed by final measurement of residual solids. The apparatus sequentially actuates the required weighing, microwave heating, solvent extraction, redrying, reweighting and calculation of the percentages of volatiles, solvent soluble sand solids.

The U.S. Pat. No. 3,909,598 discloses an apparatus and method for automatically determining weight changes in substances containing volatile material. The apparatus automatically determines the percentage of volatiles in a substance which contains unknown quantities of moisture and the like. A digital panel meter provides a readout coupled with the ability to recall the original weight and the weight obtained after removal of the volatile material.

For background purposes and indicative of the art to which the invention relates, reference may be made to the following remaining patents found in the patent search.

| PAT. NO. | INVENTOR | ISSUED |
|---|---|---|
| 3,673,852 | Davis | July 1972 |
| 3,890,825 | Davis | June 1975 |
| 4,398,835 | Athey et al | August 1983 |
| 4,753,889 | Collins | June 1988 |

### DISCLOSURE OF THE INVENTION

The invention discloses a system with two embodiments for determining the percent fat of a meat product. In its most basic design, the system consists of:

a) means for determining the percent moisture of a pre-heated wet meat product sample,

b) entering the percent moisture of the wet sample into the system,

c) means for determining the percent moisture of the post-heated dry meat product sample,

d) entering the percent moisture of the dry sample into the system, and

e) calculating and displaying the percent fat content of the sample.

The system is primarily designed to determine the percent fat content of a meat product such as beef, pork and poultry. However, the percent fat content of other meat products such as ostrich, buffalo, venison, etc., can also be determined.

There are two embodiments of the system, and in order to use either, it is first necessary to determine the moisture content of the meat product. This is determined by performing a series of steps which include: weighing a sample of a wet preheated meat product to obtain a wet weight, subjecting the preheated meat product to a heating means to allow volatile solubles to be extracted, applying heat from heating the heating means until the meat product is substantially dry, and finally reweighting the meat product to obtain the dry weight. The dry weight is subtracted from the wet weight and the remainder is divided by the wet weight. The resulting quotient is then multiplied by one-hundred to obtain the percent moisture of the meat product. The percent fat content is then obtained. The above steps are either performed manually or automatically as described below.

As previously stated the system is disclosed in two embodiments. The first embodiment utilizes an integrated percent moisture and fat analyzer that encompasses within a single enclosure all the elements to automatically determine the percent moisture and fat content of a meat product. The second embodiment utilizes a mechanical design in the form of a slide chart. On one side of the slide chart the percent fat of a type of meat is shown.

In view of the above disclosure, the primary object of the invention to provide an easily operated meat product fat testing system which can quickly determine the percent fat of a meat product.

In addition to the primary object, it is also an object of the invention to provide a meat product fat testing system that:
requires no manual entries in the third design,
is easy to operate,
is portable,
is reliable and long-lasting,
can be used in homes and businesses,
is easily readable,
is cost effective from both a manufacturer's and consumer's point of view.

These and other objects and advantages of the present invention will become apparent from the subsequent detailed description of the preferred embodiment and the appended claims taken in conjunction with the accompanying drawings.

### BRIEF DESCRIPTION OF THE DRAWINGS

FIG. 1 is a front orthographic view of a meat product fat testing system's (MPFTS) fat testing/analyzing unit.

FIG. 2 is a top orthographic view of an upper section with a hinged lid raised/opened, exposing meat product weighing means

FIG. 3 is a front orthographic view of the MPFTS's fat testing/analyzing unit with a horizontal surface attached thereto.

FIG. 4 is a front orthographic view of the MPFTS's fat testing/analyzing unit with securing bolts extending downward from the unit's lower surface.

FIG. 5 is an orthographic view of a hand-held calculating unit with a stationary member and a movable member partly extracted.

FIG. 6 is a top view of the stationary member showing spacers and the movable member.

FIG. 7 is an elevational view of the front side of the movable member.

FIG. 8 is a front orthographic view of the hand-held calculating unit comprising of an electronic calculator.

FIG. 9 is an elevational view of a sample spreader.

FIG. 10 is a top plan view of a heat and drip resistant testing sample holder (HDRTSH) that is used to hold a meat product sample within the fat testing/analyzing unit.

### BEST MODE FOR CARRYING OUT THE INVENTION

The mode for carrying out the invention is presented in terms that disclose a preferred embodiment and a second embodiment of a meat product fat testing system (MPFTS 10). For many food products it is necessary and/or desirable to know information about the quality and positive or negative health characteristics of the product. This is especially pertinent for meat products. One of the most important characteristics of a meat product is the percent fat content. Suppliers, retailers and even consumers often require the percent fat content of a meat product.

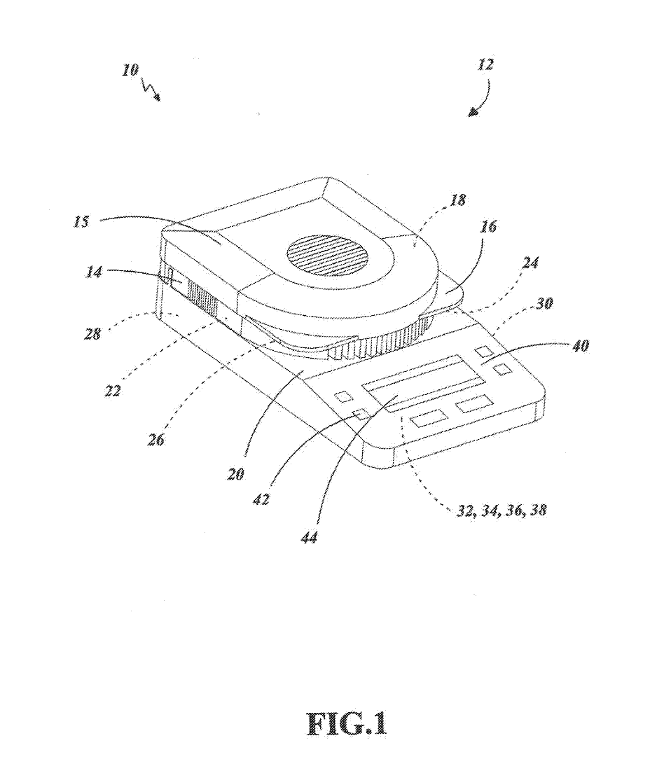

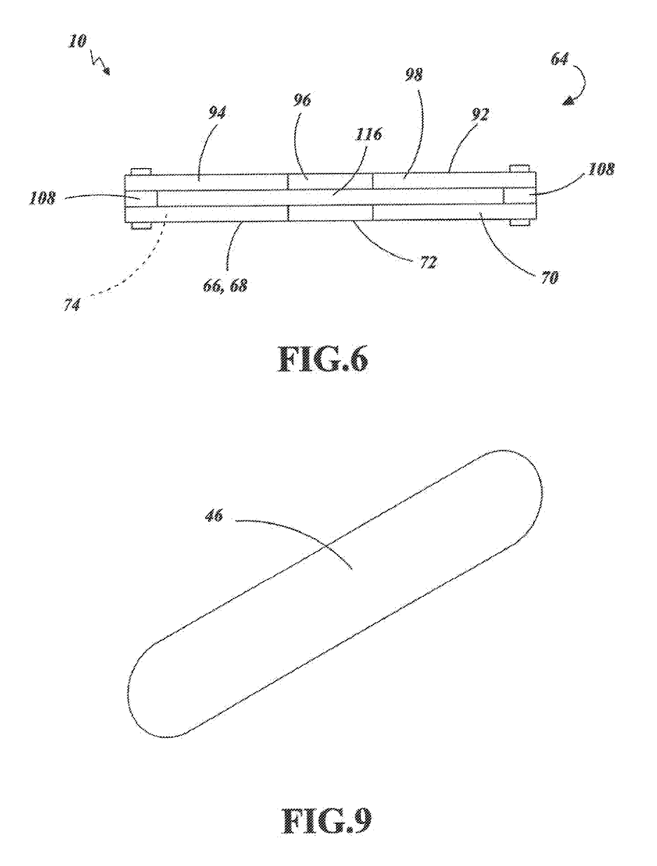

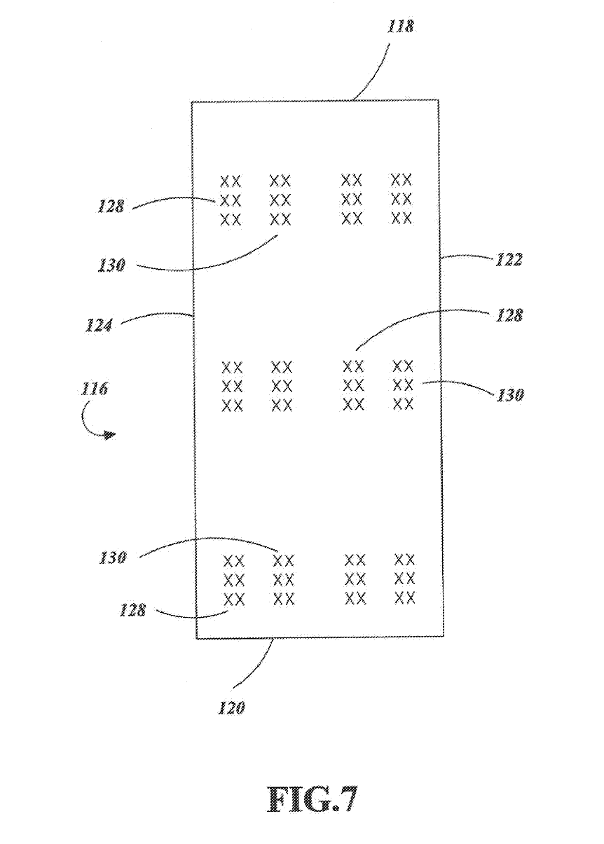

The MPFTS 10 provides a person with the ability to quickly and easily determine the percent fat content of a meat product. The MPFTS 10, as shown in FIGS. 1-10, is primarily used to determine the percent fat content of beef, pork or poultry. Other meat products, such as ostrich, buffalo or venison can also be tested/analyzed for percent fat content.

The first embodiment of the MPFTS 10, as shown in FIGS. 1-4, is comprised of a fat testing/analyzing unit 12, which is an integral device that quickly and easily tests/analyzes a sample of a meat product to determine the percent fat content of the product. As part of the test protocol, the unit 12 also determines the percent moisture content of the meat product sample.

The fat testing/analyzing unit 12 is comprised of an upper section 14 having a hinged lid 15 with a hand grip 16 for raising and lowering the lid 15 and an opening 17, and a lower section 20. As a result of the functionality, the upper section 14 is known as a heating chamber, and located within the upper section 14 is a heating means 18 which is preferably a non-glass electric heating device, although a halogen bulb or any other heat source such as infrared unit can also be utilized.

Located at the top of the lower section 20 is a meat product weighing means 24, which is preferably an electronic scale; and electronic components comprising a fat/moisture calculator 30 that is operated by a microcontroller 32 including a buffer 34, memory 36 and firmware 38.

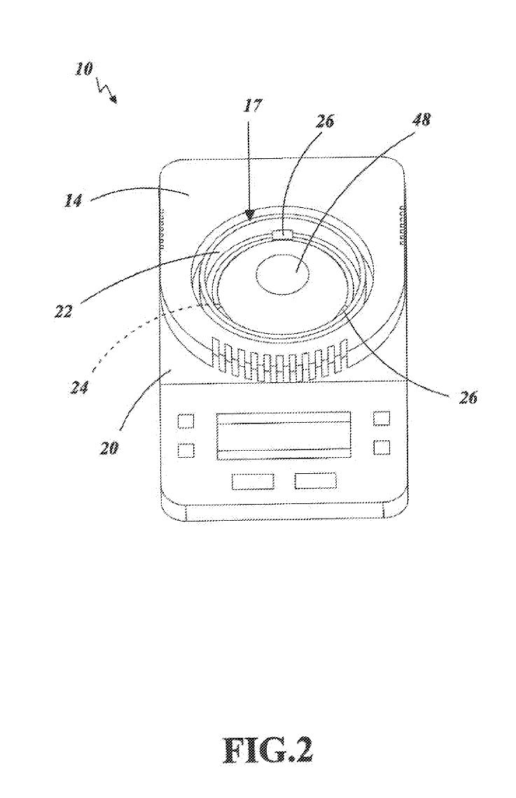

As also shown in FIG. 2, the tray 22 is placed below a tri-spoked structure and functions as a liner for the interior of the lower section. Although a tri-spoked structure 26 is shown, other structures including multi-spoked or non-spoked can be effectively used. It should be noted, the tray 22 is not a component of the meat product weighing means 24 and is not attached to the weighing means 24, rather the tray 22 is utilized as a clean-out tray in case of inadvertent spillage of the meat product sample, which could affect the performance of the weighting means. The meat product is applied/spread on a heat and drip resistant testing sample holder, as described below, and is then covered by a microfiber glass disc (not shown) or other similar covering means. Although FIGS. 1 and 2 show a U-shaped upper section and lid extending upward from the lower section 20. As can be seen, the tray 22 and the tri-spoked structure 26 are situated in the opening 17 within the upper section 14. While this particular U-shaped upper section structural design as shown is effective, the fat testing/analyzing unit 12 can include other structural designs, either extending upward or non-extending. As long as the fat testing/analyzing unit 12 is capable of housing the required components and performing the required steps, the shape and size of the unit 12 is completely variable.

On an outer surface of the lower section 20 is a panel 40 comprising controls 42 for activating/operating the unit 12 and a display 44 that shows the calculated percent fat content of the meat product sample. A timer 28 functions in combination with the heating means 26 to provide a programmed stand-by temperature for pre-heating the meat product sample prior to testing/analysis. The timer 28 is also used to produce incremental heat and to apply an optimal heat cut-off time, thereby reducing the time required to perform testing/analysis on a meat product sample.

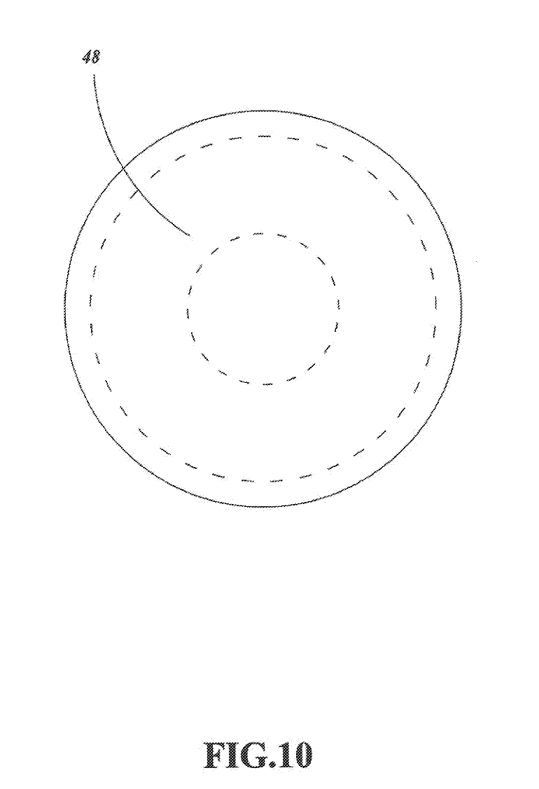

A meat product sample is placed on a sample testing holder and either evenly spread by use of a person's finger(s), or a sample spreader 46 which is preferably shaped as an elongated strip, as shown in FIG. 9, can be utilized. The benefits of using the sample spreader 46 are that it is easier to spread the sample on the sample holder and more hygienic since a new sample spreader 46 can be used for each sample that is tested/analyzed. Also, in lieu of a generic testing sample holder, a heat and drip resistant testing sample holder (HDRTSH) 48 can be used. The HDRTSH 48 is an invention by the same applicant as the instant application and is currently patent pending. The HDRTSH 48, as shown alone in FIG. 10, allows a person to spread a meat product sample on a substrate that is heat and drip resistant, as shown in FIG. 2. The benefit is that the HDRTSH is disposable after use, which negates the requirement of cleaning a sample holder after each use.

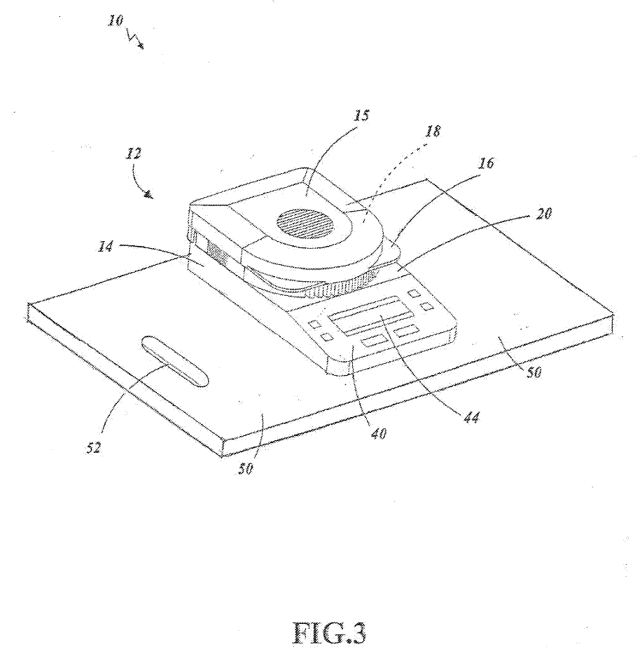

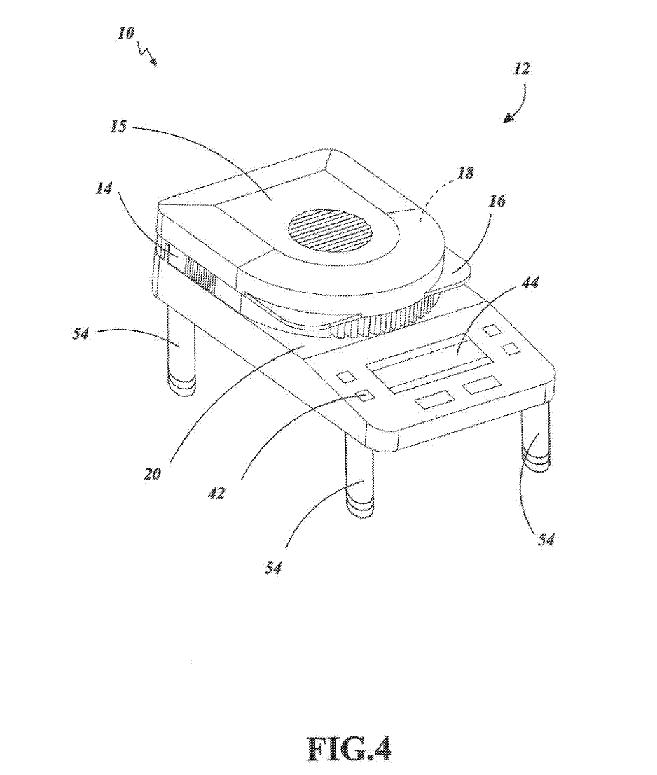

In addition to the use of the sample spreader 46 and HDRTSH 48, there are other improvements over prior art fat testing/analyzing units. As shown in FIG. 3, a horizontal surface 50, preferably with at least one grip opening 52, can be attached to the unit 12. The horizontal surface 50, which can be similar to a cutting board, provides a location where a meat product sample can be prepared prior to testing/analyzing. The horizontal surface 50 can be made of metal, plastic or wood and offers a better location for sample preparation than a counter top which is typically the location for sample preparation and is prone to dirt and other contaminants that could come in contact with the sample and may affect the test results. The unit 12 is shown in the center of the horizontal surface, but the unit 12 can also be placed on either the right side or the left side. The horizontal surface 50 is preferably attached to the bottom of the unit by bolts or screws, and multiple bolt or screw holes can be utilized. Additionally, as shown in FIG. 4, at least two, and preferably four, bolts 54 can extend downward from a lower surface of the unit 12. The bolts 54 allow the unit 12 alone to be securely mounted at a desired/optimal location, and prevent the unit 12 from being moved, stolen or dropped. Bolts can also extend downward from the horizontal surface 50 to allow a unit 12 and the horizontal surface 50 to be mounted together.

In order to use the fat testing/analyzing unit 12 to determine the percent fat content of a meat product, the following steps are performed:

a) acquire a sample of a meat product,

b) raise a hinged upper section of said fat testing/analyzing unit, thereby exposing a meat product weighing means,

c) tare to zero the weight of the HDRTSH and a covering microfiber glass disc, place the meat product sample on the HDRTSH that is on a tri-spoked structure on the sample tray and cover with the microfiber glass disc,

d) lower the hinged upper section,

e) use controls on said fat testing/analyzing unit to being testing/analyzing protocols, which include initially weighting the meat product sample, applying heat from a heating means within said unit's upper section, weigh meat product sample post-heating, calculate percent fat content of the sample by use of an internal microcontroller and firmware within said unit,

f) read the percent fat content of the meat product sample from a display on said fat testing/analyzing unit, and

g) remove and discard the meat product sample from said fat testing/analyzing unit.

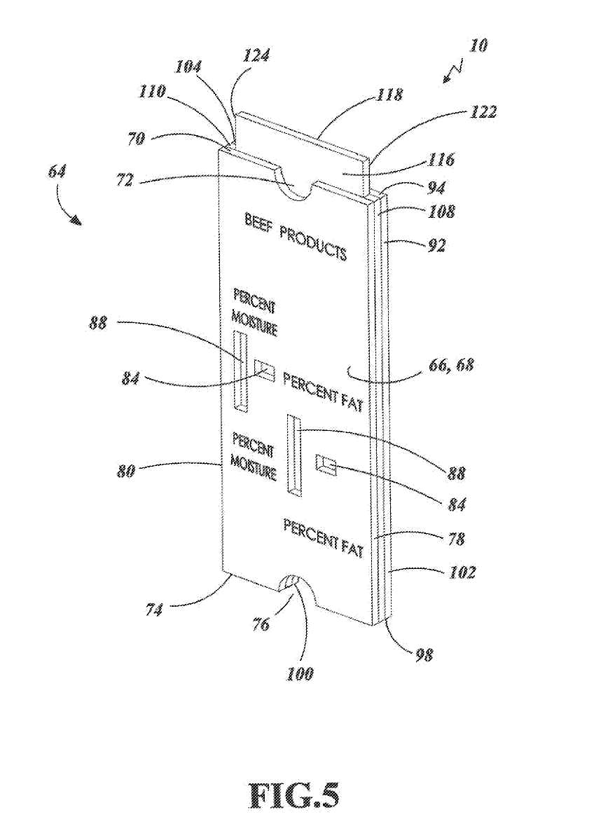

The second embodiment of the MPFTS 10 is comprised of the hand-held calculating unit 64 which is used in combination with a conventional heat source and a scale. As shown in FIGS. 5-7, the hand-held calculating unit 64 is comprised of a stationary member 66 and a movable member 116. The stationary member 66 has a front surface 68 comprising an upper edge 70 having a half-circle slot 72, a lower edge 74 having a half-circle slot 76, a right edge 78, a left edge 80, a percent fat window 84, and a percent moisture window 88. A rear surface 92 comprises an upper edge 94 having a half-circle slot 96, a lower edge 98 having a half-circle slot 100, a right edge 102, and a left edge 104. The half-circle slots on the rear surface 92. A spacer 108 is located between the front surface 68 and the rear surface 92, thereby creating a space between the surfaces and an upper longitudinal opening 110.

The movable member 116, as shown in FIGS. 5-7, is located within the space between the stationary member's front and rear surfaces 68,92. The movable member 116 comprises an upper edge 118, a lower edge 120, a right edge 122, a left edge 124, percent fat FIG. 128, and percent moisture FIG. 130. As previously disclosed, the hand-held calculating unit 64 is used in combination with a conventional heat source and scale, which are utilized to obtain pre-heated wet and post-heated dry weight readings of a sample of a meat product. The respective moisture readings are input into the unit 64, and by manually moving the movable member 116 based on the moisture readings, the percent fat content FIG. 128 of the meat product sample is displayed in the percent fat window 84, and the percent moisture content FIG. 130 is displayed in the percent moisture window 88 on the stationary member's front surface 68.

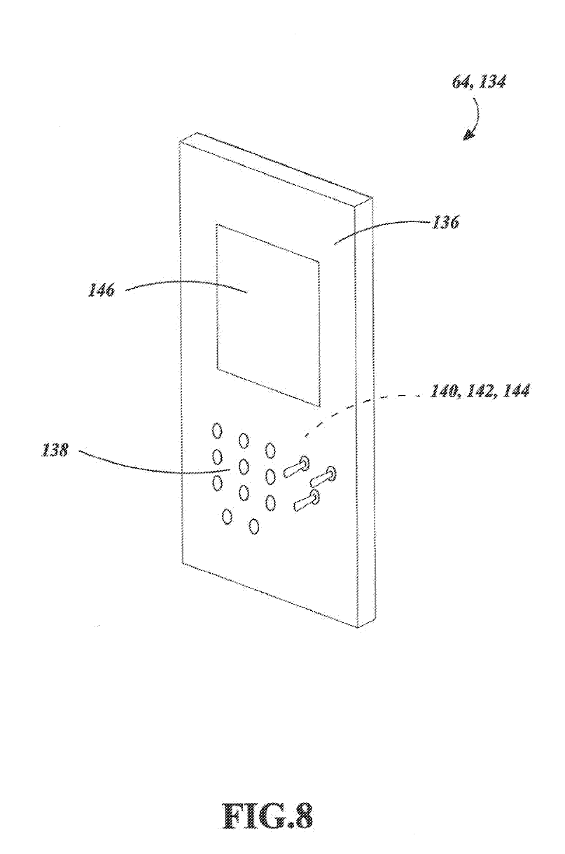

As shown in FIG. 8, the hand-held calculating unit 64 can also be comprised of an electronic calculator 134 comprising a housing 136, a keypad 138, a microcontroller 140, memory 142, firmware or software 144, and a display 146. The electronic calculator 134 performs the same fat/moisture testing and analysis of a meat product sample as the hand-held calculating unit 64, but instead of manually functioning, the electronic calculator uses electronic means to produce the displayed results after the wet and dry moisture readings are input into the calculator via the keypad 138. Alternately, the electronic calculator 134 can be comprised of an application (APP, not shown) on a wireless device such as a smartphone, which could utilize wireless or wired functionality. Additionally, both the fat testing/analyzing unit 12 and the electronic calculator 134 can include a digital output, such as USB or RS232, that facilitates interface between the unit 12 or calculator 134, and an electronic device including a computer, printer, mobile device or other electronic device.

While the invention has been described in detail and pictorially shown in the accompanying drawings it is not to be limited to such details, since many changes and modification may be made to the invention without departing from the spirit and the scope thereof. Hence, it is described to cover any and all modifications and forms which may come within the language and scope of the claims.

1. A meat product fat testing system (MPFTS) for determining the percent fat content of a meat product, wherein said MPFTS is comprised of two embodiments: a fat testing/analyzing unit and a hand-held calculating unit, wherein said fat testing/analyzing unit integrally houses a heating means, a meat product weighing means, a fat/moisture analyzer, a microcontroller and a display, wherein a sample of a meat product is placed into said fat testing/analyzing unit, said unit is activated, and the percent fat content of the meat product sample is automatically analyzed and the results shown on said unit's display, wherein said hand-held calculating unit is used in combination with a conventional scale and a heat source which are utilized to obtain pre-heated wet and post-heated dry weight readings of a sample of a meat product, wherein the weight readings are input into said calculating unit which displays in a window the percent fat content of the meat product sample.

2. The meat product fat testing system as specified in claim 1 wherein said fat testing/analyzing unit's heating means is comprised of a non-glass heating element.

3. The meat product fat testing system as specified in claim 1 wherein said fat testing/analyzing unit determines an end-time of the percent fat content test/analysis, thereby reducing the time required for a single test/analysis.

4. The meat product fat testing system as specified in claim 1 wherein said fat testing/analyzing unit further comprising a timer that functions in combination with the heating means to provide a programmed stand-by temperature for pre-heating the meat product sample prior to testing/analysis.

5. The meat product fat testing system as specified in claim 1 wherein said timer further allows the heating means to produce incremental heat and to determine and apply an optimal heat cut-off time, thereby reducing the time required to perform a testing/analysis on a meat product sample.

6. The meat product fat testing system as specified in claim 1 wherein said fat testing/analyzing unit further comprises at least two bolts that extend through said enclosure into a surface beneath said enclosure to secure the placement/location of said enclosure.

7. The meat product fat testing system as specified in claim 1 wherein said fat testing/analyzing unit further comprises a horizontal surface that is utilized for meat product sample preparation prior to testing/analyzing, and as a support surface for said enclosure, wherein the horizontal surface further comprises at least one handle for lifting or altering the position of the horizontal surface.

8. The meat product fat testing system as specified in claim 1 wherein said fat testing/analyzing unit further comprises a sample spreader in the form of an elongated strip that facilitates the placement of a meat product sample in said unit.

9. A meat product fat testing system (MPFTS) for determining the percent fat content of a meat product, wherein said MPFTS is comprised of two embodiments: a fat/moisture analyzing unit and a hand-held calculating unit, wherein said first embodiment is comprised of:
A. the fat/moisture analyzing unit comprising:
1) an upper section having a hinged lid with a hand grip, and an opening,
2) a lower section having a meat product weighing means, a panel, a fat/moisture calculator, a display, controls and electronic components including a buffer, memory, firmware, a microcontroller, and
3) heating means located within said upper section, wherein a sample of meat product is placed on a testing sample holder that is on a testing sample holder located on said weighing means and is accessible via the opening that is exposed when said lid is raised, and a microfiber glass disc is then placed over the meat product sample, wherein a series of steps are performed including weighing the meat product sample when wet, utilizing said heating means to remove moisture from the meat product sample, weighing the meat product sample when “dry”, and calculating the percent fat and percent moisture content from the pre-heated and post-heated samples, wherein the percent content(s) of the meat product is shown on the display, wherein the tested meat product sample is removed and discarded, thereby allowing a subsequent test to be performed on another meat product sample, wherein said second embodiment is comprised of:
B. the hand-held calculating unit comprising:
1) a stationary member with a front surface comprising an upper edge having a half-circle slot, a lower edge having a half-circle slot, a right edge, a left edge, a percent fat window and a percent moisture window, and a rear surface comprising an upper edge having a half-circle slot, a lower edge having a half-circle slot, a right edge, and a left edge, wherein a spacer is located between said font surface and said rear surface, thereby creating a space between said surfaces and an upper longitudinal opening, and
2) a movable member that is located within the space between said stationary member's front surface and rear surface, wherein said movable member is comprised of an upper edge, a lower edge, a right edge, a left edge, percent fat figures, and percent moisture figures, wherein said hand-held calculating device is used in combination with a conventional scale and a heat source which are utilized to obtain pre-heated wet and post-heated dry weight readings of a sample of a meat product, wherein the respective moisture readings are input into said hand-held calculating unit, and by manually moving said movable member based on the moisture readings, the percent fat content figure of the meat product sample is displayed in the percent fat window.

10. The meat product fat testing system as specified in claim 9 wherein the meat product is selected from the group consisting of beef, poultry, pork, ostrich, buffalo and venison.

11. The meat product fat testing system as specified in claim 9 wherein said fat testing/analyzing unit's heating means is comprised of a non-glass electric heating element.

12. The meat product fat testing system as specified in claim 9 wherein said fat testing/analyzing unit's heating means is comprised of a halogen bulb.

13. The meat product fat testing system as specified in claim 9 wherein said fat testing/analyzing unit determines an end-time of the percent fat content test/analysis, thereby reducing the time required for a single test/analysis.

14. The meat product fat testing system as specified in claim 9 wherein said fat/moisture analyzing unit's timer functions in combination with said heating means to provide a programmed stand-by temperature for pre-heating the meat product sample prior to testing/analysis, and to produce incremental heat that is used to determine and apply an optimal heat cut-off time, thereby reducing the time required to perform testing/analysis on a meat product sample.

15. The meat product fat testing system as specified in claim 9 wherein said fat testing/analyzing unit further comprises at least two bolts that extend from a lower surface of said unit's lower section into a surface beneath said enclosure to secure the placement/location of said unit.

16. The meat product fat testing system as specified in claim 9 wherein said fat testing/analyzing unit further comprises a horizontal surface that is utilized for meat product sample preparation prior to testing/analyzing, and as a support surface for said enclosure, wherein the horizontal surface further comprises at least one grip opening for lifting or altering the position of the horizontal surface.

17. The meat product fat testing system as specified in claim 9 wherein said fat testing/analyzing unit further comprises a sample spreader in the form of an elongated strip that facilitates the placement of a meat product sample in said unit.

18. The meat product fat testing system as specified in claim 9 wherein said hand-held calculating unit is further comprised of an electronic calculator device comprising a housing, a keypad, a microcontroller, memory, firmware or software, and a display, wherein said electronic calculator is connected via a cable to said hand-held calculating unit and performs the same fat/moisture testing and analysis as said hand-held calculating unit, but uses electronic calculating means to produce the displayed fat content results after the wet and dry moisture readings are input into said calculator via the keypad.

19. A method for determining the percent fat content of a meat product by use of a fat testing/analyzing unit, wherein said method comprises the following steps:
a) acquire a sample of a meat product,
b) raise a hinged lid on an upper section of said fat testing/analyzing unit, thereby exposing meat product weighing means,
c) tare to zero the weight of the HDRTSH and a covering microfiber glass disc, place the meat product sample on the HDRTSH that is on a tri-spoked structure on the sample tray and cover with the microfiber glass disc,
d) lower the hinged upper section,
e) use controls on said fat testing/analyzing unit to being testing/analyzing protocols, which include initially weighting the meat product sample, applying heat from a heating means within said unit's upper section, weigh the meat product sample post-heating, calculate percent fat content of the sample by use of an internal microcontroller and firmware within said unit,
f) read the percent fat content of the meat product sample from a display on said fat testing/analyzing unit, and
g) remove and discard the meat product sample from said fat testing/analyzing unit.

20. The meat product fat testing system as specified in claim 19 wherein the meat product is selected from the group consisting of beef, pork, poultry, ostrich, buffalo, and venison.
